(19) United States
(12) Patent Application Publication
(10) Pub. No.: US 2026/0337683 A1
(43) Pub. Date: Oct. 29, 2026

(54) MOLECULAR SIGNATURE FOR MOLECULAR DIAGNOSIS OF OBSTRUCTIVE SLEEP APNEA

(71) Applicant: THE CURATORS OF THE UNIVERSITY OF MISSOURI, Columbia, MO (US)

(72) Inventors: Rene CORTESE, Columbia, MO (US); David GOZAL, Columbia, MO (US)

(21) Appl. No.: 19/114,215

(22) PCT Filed: Sep. 21, 2023
(86) PCT No.: PCT/US2023/033341

§ 371 Date: Mar. 21, 2025

Related U.S. Application Data
(60) Provisional application No. 63/376,697, filed on Sep. 22, 2022.

(51) Int. Cl. C12Q 1/6883

(52) U.S. Cl. CPC C12Q 1/6883; C12Q 2600/158

(57) ABSTRACT

Disclosed are methods for identifying and treating a subject having, or suspected of having, obstructive sleep apnea (OSA) based on the gene expression level of CD70, HCST, CRIPI, IL 32, EN Cl, GZMB, GZMK, HLA.DQA2, CD300A, LINC02446, DUSP2, ITGBI, LGALSI, IFIT3, CEBPD., TNFRSF4, HBB, NEATI, LTBPI, ZNF683, SOX4, SMC4, LMSI, VIM, TRGCI, MAF, S100A4, LYAR, TRDC, TRGC2, RGSI, ANXAI, and combinations thereof present in a patient sample, and diagnosing OSA in the subject if there is a measurable change in the expression level as compared to a reference sample obtained from a subject who does not have OSA.

### CROSS REFERENCE TO RELATED APPLICATIONS

This application claims priority under 35 U.S.C. 119(e) to U.S. Provisional Patent Application Ser. No. 63/376,697, filed on Sep. 22, 2022, the disclosure of which is hereby incorporated by reference in its entirety.

### STATEMENT OF GOVERNMENT SUPPORT

This invention was made with government support under AG061824 awarded by the National Institutes of Health. The government has certain rights in the invention.

### BACKGROUND

The present disclosure relates generally to medicine. More particularly, the present disclosure is directed to methods for identifying a subject having obstructive sleep apnea.

Obstructive Sleep Apnea (OSA) is the most severe phenotype of Sleep Disordered Breathing (SDB) and is highly prevalent across all age groups with an estimated prevalence of 2-10% in the general population. Over a billion adults (men and women) have mild to severe OSA worldwide. Importantly, OSA affects 1.2-5.7% of children, with a peak prevalence occurring at 2-8 years of age, which coincides with the peak age of tonsillar and adenoidal hypertrophy. OSA is characterized by recurrent events of upper airway obstruction during sleep leading to intermittent hypoxia, episodic arousals (i.e., sleep fragmentation), as well as episodic hypercapnia and increased intrathoracic pressure swings. Many of the clinical characteristics of pediatric OSA, and the determinants of its epidemiology differ from those of adult OSA. Furthermore, OSA is a major cause of cardiovascular morbidity and neurocognitive dysfunction in children. Significant associations between sleep and cognitive development, temperament and behavior have been observed in infants during the first year of life and in school aged children, even when only mild SDB is present. Children with parent-reported OSA symptoms are associated with composite and domain-specific problem behaviors and alterations of brain structure, mainly in the frontal lobe.

Evidence supports that OSA promotes a low-grade inflammatory state. IL-8 levels were increased in LPS-induced ex vivo cultures of PBMCs from OSA children compared to control children. However, neither the production of other cytokines nor the cytokine expression in ex vivo cultures of Peripheral Blood Mononuclear Cells (PBMCs) from OSA children pre- and post-AT, showed statistically significant differences. Noteworthy, the cytokine production capacity in these samples showed a broad distribution among samples of the same group. For example, IL-8 expression ranged from 10,000 pg/mL to 120,000 pg/mL in both pre- and post-AT samples, suggesting that subpopulations of PBMCs may differently express this cytokine, that there is inter-individual heterogeneity, and importantly, that such heterogeneity may not be detected by bulk expression analysis.

Polysomnography (PSG) represents the golden standard for determining SDB in adults and children. PSG requires staying overnight in a laboratory, where sleep, respiratory and neurological parameters are monitored by a trained technician. PSG results are complex, and their interpretation require evaluation by a highly trained sleep physician. As such, PSG is an expensive medical procedure which represents a significant burden for the patient, insurers, and the health system at large.

Accordingly, there remains a need for alternative methods to identify subjects having OSA.

### BRIEF DESCRIPTION

In one aspect, the present disclosure is directed to a method of diagnosing obstructive sleep apnea in a subject having or suspected of having obstructive sleep apnea, the method comprising: providing a biological sample from the subject; determining a gene expression level of CD70, HCST, CRIP1, IL 32, ENC1, GZMB, GZMK, HLA.DQA2, CD300A, LINC02446, DUSP2, ITGB1, LGALS1, IFIT3, CEBPD., TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof, and diagnosing obstructive sleep apnea in the subject if there is a measurable change in the expression level of the CD70, the HCST, the CRIP1, the IL 32, the ENC1, the GZMB, the GZMK, the HLA.DQA2, the CD300A, the LINC02446, the DUSP2, the ITGB1, the LGALS1, the IFIT3, the CEBPD, the TNFRSF4, the HBB, the NEAT1, the LTBP1, the ZNF683, the SOX4, the SMC4, the LMS1, the VIM, the TRGC1, the MAF, the S100A4, the LYAR, the TRDC, the TRGC2, the RGS1, the ANXA1, and combinations thereof, as compared to a reference expression of CD70, HCST, CRIP1, IL 32, ENC1, GZMB, GZMK, HLA.DQA2, CD300A, LINC02446, DUSP2, ITGB1, LGALS1, IFIT3, CEBPD., TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof, obtained from a subject who does not have obstructive sleep apnea In another aspect, the present disclosure is directed to a biomarker panel for identifying a subject as having obstructive sleep apnea comprising: CD70, HCST, CRIP1, IL 32, ENC1, GZMB, GZMK, HLA.DQA2, CD300A, LINC02446, DUSP2, ITGB1, LGALS1, IFIT3, CEBPD., TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof.

In another aspect, the present disclosure is directed to a method of treating a subject having or suspected of having obstructive sleep apnea, the method comprising: providing a biological sample from the subject; determining a gene expression level of CD70, HCST, CRIP1, IL 32, ENC1, GZMB, GZMK, HLA.DQA2, CD300A, LINC02446, DUSP2, ITGB1, LGALS1, IFIT3, CEBPD., TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof, and diagnosing obstructive sleep apnea in the subject if there is a measurable change in the expression level of the CD70, the HCST, the CRIP1, the IL 32, the ENC1, the GZMB, the GZMK, the HLA.DQA2, the CD300A, the LINC02446, the DUSP2, the ITGB1, the LGALS1, the IFIT3, the CEBPD, the TNFRSF4, the HBB, the NEAT1, the LTBP1, the ZNF683, the SOX4, the SMC4, the LMS1, the VIM, the TRGC1, the MAF, the S100A4, the LYAR, the TRDC, the TRGC2, the RGS1, the ANXA1, and combinations thereof, as compared to a reference expression of CD70, HCST, CRIP1, IL 32, ENC1, GZMB, GZMK, HLA.DQA2, CD300A, LINC02446, DUSP2, ITGB1, LGALS1, IFIT3, CEBPD., TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof, obtained from a subject who does not have obstructive sleep apnea; and treating the subject diagnosed as having obstructive sleep apnea by administering a treatment for obstructive sleep apnea to the subject diagnosed as having obstructive sleep apnea.

### BRIEF DESCRIPTON OF THE DRAWINGS

The disclosure will be better understood, and features, aspects and advantages other than those set forth above will become apparent when consideration is given to the following detailed description thereof. Such detailed description makes reference to the following drawings, wherein:

FIGS. 1A-1D depict building and validation of a molecular signature for diagnosis of pediatric sleep apnea. FIG. 1A depicts a molecular signature consisting of 32 genes selected from single-cell transcriptional profiles corresponding to specific cell type and clinical attributes, and combined, according to sample ID. FIG. 1B depicts the training and testing datasets created using a 70:30 split to generate the observed ROC curves for each statistical method. FIG. 1C. The molecular signature showed a high performance for discriminating OSA patients from no OSA/no snoring controls, with the ROC-AUC analysis resulting in AUC of 0.93, 0.96, and 0.92 for Empirical, Binormal, and Non-parametric ROCs, respectively, with 93% and 95% PPV and NPV, respectively. FIG. 1D. the molecular signature had a high performance to distinguish between OSA patients from primary snoring individuals (AUCs=0.97, 0.99, and 0.96 for Empirical, Binormal, and Non-parametric ROCs, respectively; 96% PPV, and 95% NPV) and primary snoring individuals from No OSA/no snoring controls (AUC=0.95, 0.98, and 0.95 for Empirical, Binormal, and Non-parametric ROCs, respectively; 96% PPV, and 95% NPV).

### DETAILED DESCRIPTION

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are described below.

While the present disclosure is susceptible to various modifications and alternative forms, exemplary embodiments thereof are shown by way of example in the drawings and are herein described in detail. It should be understood, however, that the description of exemplary embodiments is not intended to limit the disclosure to the particular forms disclosed, but on the contrary, the intention is to cover all modifications, equivalents and alternatives falling within the scope of the disclosure as defined by the embodiments above and the claims below. Reference should therefore be made to the embodiments above and claims below for interpreting the scope of the present disclosure.

Unless defined otherwise, all technical and scientific terms used herein have the same meaning as commonly understood by one of skill in the art to which the invention pertains. Although any methods and materials similar to or equivalent to those described herein can be used in the practice or testing of the present disclosure, the preferred methods and materials are described herein. Moreover, reference to an element by the indefinite article “a” or “an” does not exclude the possibility that more than one element is present, unless the context clearly requires that there be one and only one element. The indefinite article “a” or “an” thus usually includes “at least one.” The terms “comprising”, “including” and “having” are intended to be inclusive and mean that there may be additional elements other than the listed elements. The term “about” means up to ±10%.

As used herein, “a subject” refers to a subset of individuals who are susceptible to, or at elevated risk of, experiencing obstructive sleep disorder. A subject can be susceptible to, or at elevated risk of, experiencing symptoms due to family history, age, environment, and/or lifestyle. Based on the foregoing, because some of the methods embodiments of the present disclosure are directed to specific subsets or subclasses of identified individuals (that is, the subset or subclass of subjects having or suspected of having obstructive sleep apnea), not all individuals will fall within the subset or subclass of individuals as described herein for certain diseases, disorders or conditions. The term “subject” includes both human subjects and animal subjects. Particularly suitable human subjects include pediatric human subjects, adolescent human subjects and adult human subjects. As used herein, “pediatric human subject” refers to a human subject ranging in age from about 2 years old to about 9 years old. As used herein, “adolescent human subject” refers to a human subject having an age of about 10 years old to about 19 years old. As used herein, “adult human subject” refers to a human subject having an age of 19 and older.

As used herein, “susceptible” and “at risk” refer to having little resistance to a certain disease, disorder or condition, including being genetically predisposed, having a family history of, and/or having symptoms of the disease, disorder or condition.

As used herein, “treating” (or “treat” or “treatment”) refers to processes involving a slowing, interrupting, arresting, controlling, stopping, reducing, or reversing the progression or severity of an existing symptom, disorder, condition, or disease, but does not necessarily involve a total elimination of all disease-related symptoms, conditions, or disorders associated with administration of the therapy.

As used herein, the term “biomarker” refers to any molecule or group of molecules found in a biological sample that can be used to characterize the biological sample or a subject from which the biological sample is obtained. For example, a biomarker may be a molecule or group of molecules whose presence, absence, or relative abundance is: characteristic of a particular cell or tissue type or state; and/or characteristic of a particular pathological condition or state; and/or indicative of the severity of a pathological condition, the likelihood of progression or regression of the pathological condition, and/or the likelihood that the pathological condition will respond to a particular treatment. As another example, the biomarker may be a cell type or a substituent molecule or group of molecules thereof. Biomarkers provided herein can be diagnostic biomarkers that can be used to detect and/or confirm the presence of obstructive sleep apnea. Biomarkers provided herein can also be monitoring biomarkers that can be serially analyzed to assess the status of obstructive sleep apnea. Biomarkers provided herein can also be pharmacodynamic biomarkers that can be used to determine a patient's response to treatment of obstructive sleep apnea. Biomarkers provided herein can also be predictive biomarkers that can be used to predict or identify an individual or group of individuals more likely to experience a favorable or unfavorable effect from treatment. Biomarkers provided herein can also be safety biomarkers that are measured before and/or after treatment to indicate the likelihood, presence, or extent of a toxicity to treatment. Biomarkers provided herein can also be prognostic biomarkers to identify obstructive sleep apnea progression and/or recurrence. Biomarkers provided herein can also be susceptibility/risk biomarkers that can indicates the potential for an individual to develop obstructive sleep apnea but who has not been diagnosed as having obstructive sleep apnea. Biomarkers provided herein can also be surrogate biomarkers that explain the clinical outcome following treatment.

A change in the expression can be an increase in the expression in a second (or subsequent) sample as compared to the expression in a first sample. A change in the expression can also be a decrease in a second (or subsequent) sample as compared to the expression in a first sample. The change in expression level in the sample(s) obtained from the subject administered a treatment can further be compared to one of a gene expression level in a sample(s) obtained from a healthy subject (a subject who is not suspected of having or has obstructive sleep apnea) and a gene expression level in a sample(s) obtained from a subject having or suspected of having obstructive sleep apnea who is not administered a treatment.

As used herein, “expression level of a biomarker” refers to the process by which a gene product is synthesized from a gene encoding the biomarker as known by those skilled in the art. The gene product can be, for example, RNA (ribonucleic acid) and protein. Expression level can be quantitatively measured by methods known by those skilled in the art such as, for example, northern blotting, amplification, polymerase chain reaction, microarray analysis, tag-based technologies (e.g., serial analysis of gene expression and next generation sequencing such as whole transcriptome shotgun sequencing or RNA-Seq), Western blotting, enzyme linked immunosorbent assay (ELISA), and combinations thereof.

As used herein, “a reference expression level” of a biomarker refers to the expression level of a biomarker established for a subject without obstructive sleep apnea, expression level of a biomarker in a normal/healthy subject without obstructive sleep apnea as determined by a medical professional and/or research professional using established methods as described herein, and/or a known expression level of a biomarker obtained from literature. The reference expression level of the biomarker can also refer to the expression level of the biomarker established for any combination of subjects such as a subject without obstructive sleep apnea, expression level of the biomarker in a normal/healthy subject without obstructive sleep apnea, and expression level of the biomarker for a subject without obstructive sleep apnea at the time the sample is obtained from the subject, but who later exhibits obstructive sleep apnea. The reference expression level of the biomarker can also refer to the expression level of the biomarker obtained from the subject to which the method is applied. As such, the change within a subject from visit to visit can indicate an increased or decreased risk for obstructive sleep apnea. For example, a plurality of expression levels of a biomarker can be obtained from a plurality of samples obtained from the same subject and used to identify differences between the pluralities of expression levels in each sample. Thus, in some embodiments, two or more samples obtained from the same subject can provide a gene expression level(s) of a biomarker and a reference expression level(s) of the biomarker.

“Obstructive Sleep Apnea” as used herein, refers to a condition that is characterized by a history of habitual snoring, and which is associated with repeated events of partial or complete obstruction of the upper airway during sleep. Obstructive Sleep Apnea (OSA) is a clinically complex syndrome affecting up to 3% of pre-pubertal children and causes subjects to experience both daytime and nighttime symptoms. As noted, the main nocturnal system is habitual snoring. However, subjects afflicted with OSA frequently experience further nocturnal symptoms including respiratory pauses, noisy breathing sounds, paradoxical breathing movements, cyanosis, restless sleep, excessive diaphoresis, sleep enuresis, as well as recurring episodes gas exchange abnormalities, such as hypercapnia and hypoxemia, and frequent arousals (i.e., sleep fragmentation). As such, OSA is typically ranked at the severe end of the clinical spectrum of sleep disordered breathing.

As used herein, the terms “correlated” and “correlating,” in reference to the use of diagnostic and prognostic biomarkers, refers to comparing the presence or quantity of the biomarker in a subject to its presence or quantity in subjects known to suffer from a given condition (e.g., OSA); or in subjects known to be free of a given condition, i.e. “normal individuals.”

In one aspect, the present disclosure is directed to a method of diagnosing obstructive sleep apnea in a subject having or suspected of having obstructive sleep apnea. The method includes providing a biological sample from the subject; determining a gene expression level of CD70, Hematopoietic Cell Signal Transducer (HCST), Cysteine Rich Protein 1 (CRIP1), Interleukin 32 (IL 32), Ectodermal-Neural Cortex 1 (ENC1), Granzyme B (GZMB), Granzyme K (GZMK), Major Histocompatibility Complex, Class II, DQ Alpha 2 (HLA.DQA2), CD300A, Long Intergenic Non-Protein Coding RNA 2446 (LINC02446), Dual Specificity Phosphatase 2 (DUSP2), Integrin Subunit Beta 1 (ITGB1), Galectin 1 (LGALS1), Interferon Induced Protein With Tetratricopeptide Repeats 3 (IFIT3), CCAAT Enhancer Binding Protein Delta (CEBPD), Tumor Necrosis Factor (TNF) Receptor Superfamily Member 4 (TNFRSF4), Hemoglobin Subunit Beta (HBB), Nuclear Paraspeckle Assembly Transcript 1 (NEAT1), Latent Transforming Growth Factor Beta Binding Protein 1 (LTBP1), Zinc Finger Protein 683 (ZNF683), Sex Determining Region Y (SRY)-Box Transcription Factor 4 (SOX4), Structural Maintenance Of Chromosomes 4 (SMC4), LIM-Zinc Finger Domain Containing (LIMS1), Vimentin (VIM), T Cell Receptor Gamma Constant 1 (TRGC1), Musculoaponeurotic Fibrosarcoma (MAF BZIP) Transcription Factor (MAF), S100 Calcium Binding Protein A4 (S100A4), Ly1 Antibody Reactive (LYAR), T Cell Receptor Delta Constant (TRDC), T Cell Receptor Gamma Constant 2 (TRGC2), Regulator Of G Protein Signaling 1 (RGS1), Annexin A1 (ANXA1), and combinations thereof, and diagnosing obstructive sleep apnea in the subject if there is a measurable change in the expression level of the CD70, the HCST, the CRIP1, the IL 32, the ENC1, the GZMB, the GZMK, the HLA.DQA2, the CD300A, the LINC02446, the DUSP2, the ITGB1, the LGALS1, the IFIT3, the CEBPD, the TNFRSF4, the HBB, the NEAT1, the LTBP1, the ZNF683, the SOX4, the SMC4, the LIMS1, the VIM, the TRGC1, the MAF, the S100A4, the LYAR, the TRDC, the TRGC2, the RGS1, the ANXA1, and combinations thereof, as compared to a reference expression of CD70, HCST, CRIP1, IL 32, ENC1, GZMB, GZMK, HLA.DQA2, CD300A, LINC02446, DUSP2, ITGB1, LGALS1, IFIT3, CEBPD., TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof, obtained from a subject who does not have obstructive sleep apnea.

Suitable subjects having or suspected of having obstructive sleep apnea include a human subject. The human subject can be a pediatric human subject, an adolescent human subject, and an adult human subject.

Suitable biological samples include whole blood. A particularly suitable biological sample includes a peripheral blood sample. A particularly suitable biological sample includes peripheral blood mononuclear cells. A particularly suitable biological sample includes RNA obtained from the peripheral blood mononuclear cells. The method can further include isolating peripheral blood mononuclear cells and subjecting the peripheral blood mononuclear cells to cell sorting. The method can further include analyzing the peripheral blood mononuclear cells with single cell RNA sequencing, polymerase chain reaction (e.g., quantitative real-time PCR), targeted RNA sequencing, microarray analysis, and combinations thereof. The method can further include determining a cell subtype wherein the cell subtypes include a T-cell subtype, a myeloid subtype, a B-cell subtype, and combinations thereof. The myeloid subtype can further be analyzed to identify classical monocytes, non-classical monocytes, platelets, dendritic cells, plasmacytoid dendritic cells, and combinations thereof in the myeloid subtype. The B-cell subtype can further be analy zed to identify naïve B-cells, memory B-cells, plasma B-cells, atypical B-cells, and combinations thereof in the B-cell subtype. Whole blood, peripheral blood, and peripheral blood mononuclear cells can be analyzed for gene expression using amplification (e.g., qPCR), targeted sequencing, and other methods for detecting gene expression.

The method can further include performing Polysomnography (PSG) on the subject having or suspected of having obstructive sleep apnea.

The method can further include determining Apnea Hypopnea Index for the subject having or suspected of having obstructive sleep apnea.

The method can further include administering a treatment for obstructive sleep apnea to the subject diagnosed as having obstructive sleep apnea. Suitable treatments include a lifestyle change (e.g., weight loss, regular exercise, moderate alcohol consumption, smoking reduction/cessation, a nasal decongestant, an allergy medication, change in sleeping position, avoidance of sedative medications), positive airway pressure (e.g., continuous positive airway pressure “CPAP”; autotitrating pressure “APAP”; bilevel positive airway pressure “BPAP”), an oral appliance, and surgery.

In another aspect, the present disclosure is directed to a method of treating obstructive sleep apnea in a subject having or suspected of having obstructive sleep apnea. The method includes providing a biological sample from the subject; determining a gene expression level of CD70, HCST, CRIP1, IL 32, ENC1, GZMB, GZMK, HLA.DQA2, CD300A, LINC02446, DUSP2, ITGB1, LGALS1, IFIT3, CEBPD., TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof, and diagnosing obstructive sleep apnea in the subject if there is a measurable change in the expression level of the CD70, the HCST, the CRIP1, the IL 32, the ENC1, the GZMB, the GZMK, the HLA.DQA2, the CD300A, the LINC02446, the DUSP2, the ITGB1, the LGALS1, the IFIT3, the CEBPD, the TNFRSF4, the HBB, the NEAT1, the LTBP1, the ZNF683, the SOX4, the SMC4, the LMS1, the VIM, the TRGC1, the MAF, the S100A4, the LYAR, the TRDC, the TRGC2, the RGS1, the ANXA1, and combinations thereof, as compared to a reference expression of CD70, HCST, CRIP1, IL 32, ENC1, GZMB, GZMK, HLA.DQA2, CD300A, LINC02446, DUSP2, ITGB1, LGALS1, IFIT3, CEBPD., TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof, obtained from a subject who does not have obstructive sleep apnea; and treating the subject diagnosed as having obstructive sleep apnea by administering a treatment for obstructive sleep apnea to the subject diagnosed as having obstructive sleep apnea.

The method can further include determining a gene expression level of CD70, HCST, CRIP1, IL 32, ENC1, GZMB, GZMK, HLA.DQA2, CD300A, LINC02446, DUSP2, ITGB1, LGALS1, IFIT3, CEBPD., TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof, after treatment; and determining if there is a measurable change in the expression level of the CD70, the HCST, the CRIP1, the IL 32, the ENC1, the GZMB, the GZMK, the HLA.DQA2, the CD300A, the LINC02446, the DUSP2, the ITGB1, the LGALS1, the IFIT3, the CEBPD, the TNFRSF4, the HBB, the NEAT1, the LTBP1, the ZNF683, the SOX4, the SMC4, the LMS1, the VIM, the TRGCI, the MAF, the S100A4, the LYAR, the TRDC, the TRGC2, the RGS1, the ANXA1, and combinations thereof, as compared to expression of CD70, HCST, CRIP1, IL 32, ENC1, GZMB, GZMK, HLA.DQA2, CD300A, LINC02446, DUSP2, ITGB1, LGALS1, IFIT3, CEBPD., TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof before treatment.

The method can further include providing a peripheral blood mononuclear cell sample from the subject; determining an amount of a cell subtype selected from a T-cell subtype, a myeloid subtype, and a B-cell subtype, and combinations thereof in the peripheral blood mononuclear cell sample. The method can further include analyzing RNA obtained from the peripheral blood mononuclear cells with single cell RNA sequencing, polymerase chain reaction (e.g., quantitative real-time PCR), targeted RNA sequencing, microarray analysis, and combinations thereof.

Suitable treatments include a lifestyle change (e.g., weight loss, regular exercise, moderate alcohol consumption, smoking reduction/cessation, a nasal decongestant, an allergy medication, change in sleeping position, avoidance of sedative medications), positive airway pressure (e.g., continuous positive airway pressure “CPAP”; autotitrating pressure “APAP”; bilevel positive airway pressure “BPAP”), an oral appliance, and surgery.

The methods can further include analyzing clinical metrics including polysomnography and Apnea Hypopnea Index for the subject having or suspected of having obstructive sleep apnea.

In one aspect, the present disclosure is directed to a biomarker panel for diagnosing obstructive sleep apnea in a subject in need thereof comprising: CD70, HCST, CRIP1, IL 32, ENC1, GZMB, GZMK, HLA.DQA2, CD300A, LINC02446, DUSP2, ITGB1, LGALS1, IFIT3, CEBPD., TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof.

### EXAMPLES

### Materials and Methods

### Patient Population and Sample Preparation

The OSA group consisted of 11 patients who were polysomnographically diagnosed with OSA (Apnea Hypopnea Index (AHI) <5 events/hour sleep). The Control group consisted of 11 asymptomatic individuals without OSA (AHI <1 events/hour sleep). Data from all sleep studies were scored using American Academy of Sleep Medicine guidelines by trained personnel who were blinded to the aims or nature of the study. OSA patients and controls were matched by age (mean ages: 6.64±1.27 and 5.73±0.59 years for OSA and Control groups, respectively; p=0.528, Student's t-test). Compared with Controls, children in the OSA groups had slightly higher Body Mass Index (BMI) (mean BMI: 22.71±2.72 and 17.92±1.95 kg/m² for OSA and Control groups, respectively; p=0.056, Student's t-test) and significantly elevated AHI (mean AHI: 16.65±2.68 and 1.25±0.13 events/hour sleep for OSA and Control groups, respectively; p=1.85×10⁻⁴, Student's t-test), in agreement with previously reported criteria. All the participants provided written informed consent and the research protocol was approved by the research ethics board at the University of Missouri (protocol ID MU_2016683). PBMCs were isolated by gradient centrifugation using Ficoll-PAQUE (GE Healthcare Pharmacia, Chicago IL), cell viability was verified, and samples were cryopreserved in liquid nitrogen until use.

### Single Cell Transcriptomics Analysis

After thawing, quality control of the isolated single-cells was conducted thoroughly to monitor the suitability of each sample before undergoing single-cell sorting and library preparation. scRNA-seq libraries were generated from isolated PBMCs using Chromium 3′ v3.1 (10X Genomics, Pleasanton, CA), with a targeted output of 10,000 cells per sample. Libraries were sequenced using an NextSeq (Illumina, San Diego, CA) instrument, targeting over 50,000 reads per cell. The dataset is available at the NCBI Gene Expression Omnibus (GEO) repository (accession number pending).

scRNAseq Data Processing

Data were processed using the manufacturer's software CellRanger (version 3.0.2). The quality of raw reads was assessed by FastQC, followed by trimming of adapters using TrimGalore, and reads that passed the raw data QC were used for mapping using STAR 63. Cell barcodes considered cells were automatically filtered by the software. Data analysis was conducted in R (version 4.1.0). Absolute unique molecular identifier (UMI) counts were normalized by converting them to relative values per 10,000 molecules per cell, then natural log transforming these values with a pseudocount of one.

### Cell Type Identification Via Cluster Analysis

Cell types were identified through an iterative, recursive process of clustering analysis followed by expert curation. All distinct cell types satisfied the criteria of having many categorically distinct gene expression features which consistently separated them from other cell types from the same patient, irrespective of the patient's disease status. Satisfaction of these conditions was demonstrated by post-hoc validation of resulting heatmaps that presented distinct marker genes for each cell type, further organized by data corresponding to each patient. Each round of cluster analysis was performed using the R package Seurat (version 4.0.5). First, feature selection of genes was performed using the Seurat implementation FindVariableGenes to select the top genes by their ranked contribution to global variance. The normalized values for these genes were then scaled using Seurat's ScaleData implementation, where the percent of mitochondrial genes from each cell was used as a variable to regress out its undesirable contribution to variance. The scaled genes were then used for principal component analysis using the Seurat's RunPCA implementation under default parameters. Feature selection of principal components was performed by taking the top ranked principal components based on the amount of variance they explained in the data. Top principal components were excluded whenever their covariate genes represented signals related to patient sex (i.e., Y-chromosome versus XIST), as well as patient-specific expression patterns irrelevant to cell type classifications (e.g., patient-restricted heat shock protein expression). Selected principal components were then used to graph-embed cells into a network based on their distances in principal component space via Seurat's FindNeighbors implementation. These networks were then used for Louvain clustering using Seurat's FindClusters implementation with a high granularity. Clusters were then carefully explored to determine the extent of within-cluster gene expression uniformity and between-cluster distinctiveness. Clusters corresponding to heterotypic cell-type multiplets are removed. Cell-populations representing similar types of cells (e.g., T and ILC lymphoid cells) were then subset from the rest of the data and the process of clustering was repeated until all multiplets were removed and new distinct cell populations were no longer resolved.

### Comparing Cell Type Composition

All cell population proportions are presented as a percentage makeup among each cell types respective cell grouping (e.g., T helper cells as a % among T/ILCs). Each value is calculated independently for each patient, patients with zero cells collected from a given cell type are considered missing values, not true zeroes. Significant differences in cell type compositions were assessed by a Wilcoxon rank-sum test performed between disease and control patient's percent values for a given cell type.

Gene Expression Correlations with Clinical Parameters

Correlations between a patient's AHI and gene expression levels for a given cell type were performed by making a “pseudo-bulk” value by averaging the gene expression values of all cells from a given cell type from the same patient. A Spearman correlation test was then used to calculate a rho and p-value in the relationship between AHI and the patient's average gene expression for the cell type being assessed.

### Interpretation of Differentially Expressed Genes and Networks

Population composition analysis was conducted using Wilcoxon rank-sum test. Correlation testing was conducted with Spearman's method. The percent makeup was visualized via loess regression (95% CI). Principal component analysis and variable plots were conducted using the factoextra package version 1.0.7. All the tests were conducted in R (version 4.1.0). Potential overlaps with biological processes and pathways were determined using Ingenuity Pathway Analysis (IPA) software (Qiagen, Valencia, CA).

### Building of Molecular Signature

Results collected from scRNAseq analytical pipeline were combined according to variables indicating the genetic symbol, cell type cluster, fold-change average after log2 transformation, and dimensional coordinates Principal Component Analysis (PCA). Package stringr (version 1.4.0) implemented in R for Statistical Computing (version 4.1.0) was used to join the gene symbols with their corresponding clusters into a single character vector for network analysis using the Pearson correlation method. Our approach affords for a more robust analysis by including the specific cell types with the metrics of genetic expression by isolating unique values using the cell-specific subtype. It is a technique for mitigating analytical conflicts associated with redundancy, (i.e., incorrectly removing duplicate values), encountered during computational modeling. Threshold for the observed correlations was initially set to 0.700, then increased to 0.875 (i.e., approximated midpoint between median and 3rd quartile). Upon completion of the correlation analysis, each of the gene and cluster variable combinations were separated, once again, into a total of four resulting columns: (a.) cluster01; (b.) gene01; and (c.) cluster02; (d.) gene02. This strategy leverages cell-type specific clusters maximizing the reach of the genetic coordinate values, (i.e., Euclidean distance) and recombining the variables according to type, or initial category (e.g., genes or clusters, ‘like-type’). Recombination of these components constrained cell-type clusters according to their paired alignment after this processing step, allowing the identification of the most relevant genetic correlations, consistent with cell type.

The molecular signature was extracted from each of the Seurat objects corresponding to specific cell type and clinical attributes, and combined, according to sample ID. Variables with greater than 70% of missing values were removed from the dataset. Remaining variables were imputed using the median.

### Molecular Signature Testing Using ROC Analysis

Empirical, binormal, and non-parametric statistical methods, each using randomForest (version 4.7.1), were the parameters leveraged in the construction of three ROC curves using R-package ROCit (version 2.1.1). Training and testing datasets were created using a 70:30 split to generate the observed ROC curves for each statistical method. After completion of this analysis, the molecular signature was determined to be the best performer of the potential biomarker panels and comprises a total of 32 genes. An additional ROC-AUC analysis was performed to validate the observed results using an independent dataset17 composed of Microarray expression values. Implementation of the ROC analysis was conducted using the molecular signature with the same computational parameters, outlined for the following groups listed: (a.) Normal vs. OSA; (b.) Normal vs. Primary snoring; and (c.) Primary snoring vs OSA.

Single Cell Transcriptional Profiling Discriminates Cellular Populations in PBMC from OSA Children and Controls

Single-cell transcriptional profiling enabled the identification of cell populations among the PBMCs. Uniform manifold approximation and projection (UMAP) demonstrated that cells clustered according to their expression profiles. Cross-referencing the UMAP results with known markers for each cell type enabled the identification of the cellular types represented in each cluster. Variations in the cell type composition were observed between the patients, and when cells were stratified according to OSA status, and OSA severity. Noteworthy, we observed heterogeneity on the single-cell expression profiles across individuals, highlighting the potential for personalization of the marker panels.

Next, differential gene expression was evaluated across the different cell types identified in PBMCs. Whereas some genes were differentially expressed in more than a cell type, some genes were unique for each population. To further explore the transcriptional profiles in each cell type, the cell clusters corresponding to specific cell populations in three subgroups were accommodated according to the cell lineages in PBMCs: i) “T-cell subgroup”, ii) “Myeloid Subgroup”, and iii) “B-cells subgroup”, and the cellular heterogeneity in each subgroup was separately assessed.

### Cellular Heterogeneity in the T-Cells Subgroup

The “T-cells subgroup” (n=76,646 cells) contained cell clusters corresponding to T-Helper (Th), T-Regulatory (Treg), T-Cytotoxic (Tc), T-gamma-delta (Tgd), Natural Killer (NK) and Innate lymphoid (ILC) cells. All identified subpopulations distinctively expressed known markers and were well represented across the samples. Single-cell transcriptional profiling revealed that OSA patients exhibited a decline in the percentage of T-helper naïve cells (Th_Naive; median percent: 28.35±2.41% and 39.62±2.23%, for the OSA and Control groups, respectively; p=0.002, Wilcoxon test). Furthermore, two cell clusters were identified whose transcriptional profiles did not correspond to any canonical T-cell population (i.e., T-Cell_X1 and T-Cell_X2). Correlation analyses revealed that AHI was significantly inversely correlated with the percentage of cells in the T-Cell_X2 (rho=−0.57, p-value=0.005; Spearman correlation test) and Th_Naive B (rho=−0.48, p-value=0.022; Spearman correlation test) groups. Furthermore, AHI was significantly directly correlated with the percentage of cells in the Tgd group (rho=0.51, p-value=0.015, Spearman correlation test), and marginally significantly directly correlated with the percentage of cells in the NK_B group (rho=0.42, p-value=0.053, Spearman correlation test).

Differentially expressed genes between the different clusters included genes whose expression was associated with known T-cell populations (e.g., CD3, CD4, FOXP3, CTL4, etc.) as well as other genes which may define previously unknown cellular populations that are characteristic among OSA patients. To illustrate the potential functional consequences of cell type specific gene expression, the biochemical pathways and molecular networks associated with genes differentially expressed in the cells identified as T-Cell_X1 and T-Cell_X2 were determined (FIGS. 2D and 2E). For example, in the T-Cell_X1 population, significant activation of two classical pathways in T-Cells: “Natural Killer Cell Signaling” (−log(p-value)=8.08), and “T Cell Receptor Signaling” (−log(p-value)=3.55), as well as the more specific inflammatory pathways “ERK/MAPK Signaling” (−log(p-value)=5.23), “Ceramide Signaling” (−log(p-value)=4.41), and “Renin-Angiotensin Signaling (−log(p-value)=3.9) was observed. In turn, in the T-Cell_X2 population, significant activation of recognized pathways for T cell activation was also observed: “T Cell Receptor Signaling” (−log(p-value)=3.51), “Signaling by Rho Family GTPases” (−log(p-value)=4.66), and “Phospholipase C Signaling” (−log(p-value)=4.46); along with activation of pathways related to immune-cell trafficking (“ILK Signaling, −log(p-value)=4.6 and “HGF Signaling” −log(p-value)=3.58) and inflammation (“WNT/β-catenin Signaling” −log(p-value)=3.99). Noteworthy, analysis of unsupervised gene networks revealed the putative activation of molecular networks specific for the T-Cell_X1 and T-Cell_X2 populations that are associated with hypoxia-inducible factor signaling and oxidative stress, two main cellular processes in OSA pathophysiology. Thus, activation of pathways and molecular networks in two previously unreported T-Cell subpopulations (i.e., T-Cell_X1 and T-Cell_X2) exhibited characteristics of T-Cell activation along with specific intracellular signaling pathways suggesting specific molecular regulation in defined cellular populations recruited by the presence of pediatric OSA.

### Cellular Heterogeneity in the Myeloid Subgroup

Recursive embedding was applied to identify cellular clusters in the “myeloid subgroup” (n=11,708) increasing the neighborhood size in the UMAP clustering. Gene expression profiles enabled the identification of cellular clusters corresponding to known myeloid cell populations: classical monocytes (cMonocytes), non-classical monocytes (ncMonocytes), platelets, dendritic cells (cDCs), and plasmacytoid dendritic cells (pDCs). Marginally to the cMonocytes cluster two clusters sharing gene expression features with platelets (cMonocyte_Platelet) or ncMonocytes (intMonocytes) were identified. Remarkably, a cluster (Monocyte_IFN) whose gene expression profiles faithfully corresponded to an IFN-primed phenotype was identified. Among genes highly expressed in the Monocyte_IFN cluster, genes were detected corresponding to the dynamin like GTPases (e.g., MX1, MX2), interferon-induced proteins (e.g., IFI44L, IFIT1, etc.), signal transducer and activator of transcription proteins (i.e., STAT1, STAT2), and 2′-5′-oligoadenylate synthetases (e.g., OAS1, OAS2, etc.). 895 genes were identified showing differential expression between the cell types, of which 674 were differentially expressed uniquely in one of the cell types: 16/58 in cMonocytes, 3/23 in ncMonocytes, 634/741 in cDC and 25/56 in pDC groups, respectively.

Analysis of cellular composition showed that the rate of cMonocytes cells was elevated in OSA patients compared with controls (average cell percentage=63.72±1.62% and 56.49±2.02% for OSA and Control groups, respectively; p=0.035, Wilcoxon test), whereas cellular composition of the other clusters was equivalent. Analysis of molecular pathways and networks revealed cell-type specific regulation induced by OSA in myeloid cells. In particular, differentially expressed genes in cMonocytes group were associated with “Production of Nitric-Oxide and Reactive Oxygen Species”, “HIF1α signaling”, and “NF-kB signaling”.

### Cellular Heterogeneity in the B-Cells Subgroup

Recursive embedding of single-cell expression profiles distinguished cluster of cells in the “B-cells subgroup” (n=17,010 cells) corresponding to Naïve B-Cells (B_Naive), Memory B-Cells (B_Memory), Plasma B-Cells (B-Plasma), and atypical B-Cells (B-Atypical). Subpopulations within the B_Memory cells were identified according to the Class-Switch Recombination (CSR) Status 22 reflecting the replacement of the immunoglobulin heavy chain constant region from IGHD/IGHM (i.e., preCSR) to IGHG/IGHA/IGHE (i.e., postCSR), with a subgroup co-expressing both classes (i.e., midCSR). In addition, two clusters of Plasma B cells (B-Plasma and B-Plasma_CC) were identified which have undergone CSR and expressed immunoglobulin at higher rates than other B cells populations. One of these populations (B-Plasma_CC) expressed high levels of transcripts related to mitosis and DNA synthesis highlighting a high rate of cell proliferation. On the other hand, the B_Atypical population consisted of a mixed cell population of CD18+/CD20+/CD21−/CD27− B-cells sharing a distinctive gene expression signature. Atypical B-Cells have been reported in response to vaccination and chronic infections 23-25 and can be also characterized according to their CSR status.

Remarkably, the rate of cells belonging to B_Atypical subpopulation was significantly higher (CV=82%) in OSA samples compared with the Control group (average cell percentage: 4.15±1.07% and 2.28±0.68% for OSA and control groups, respectively; p=0.013, Wilcoxon test). Likewise, a clear, yet marginally significant, increase in B-Memory cells (CV=25%; average cell percentage: 39.34±3.18% and 31.39±3.50% for OSA and control groups, respectively; p=0.088, Wilcoxon test) and decrease in B_Naive cells (CV=−15%; average cell percentage: 56.51±3.99% and 66.33±3.60% for OSA and control groups, respectively; p=0.076, Wilcoxon test) was observed in OSA patients compared with controls.

Principal Component Analysis (PCA) including counts for the B-cell subgroups and phenotypic variables (i.e., age, BMI and AHI) showed that OSA patients clustered separately from controls. Graph of variables revealed the AHI, and subpopulations of mature B-Cells (i.e., B_Memory, B_Atypical, B-Plasma, and B-Plasma_CC) were the variables dragging the sample distribution towards the OSA samples. Conversely, B_Naive counts dragged sample distribution towards the position of Control samples. In contrast, similar PCA analysis using counts from the T-cells and Myeloid subgroups discriminated the OSA and Control groups to a much lesser extent.

4,010 genes were identified that were differentially expressed across the B-cell subgroups, with 1,726 being differentially expressed in only one cell type: 221/256 in B_Naive, 281/700 in B_Memory, 295/520 in B_Atypical, 92/858 in B-Plasma, and 835/1676 in B-Plasma_CC. Differentially expressed genes in all B-cell types were related to canonical pathways associated with B cell development, activation, and immune cell crosstalk, although differences were noted among the different B-cell subpopulations. For example, the pathway “B cell development” was overrepresented in B_Naive, B_Memory and B_Atypical groups (−log(pvalue)=6.63, 5.44, and 12.7, respectively), but not in B_Plasma and B-Plasma_CC groups (−log(pvalue)=1.72 and 0, respectively). Comparative analysis of molecular networks involved in pathophysiological processes related to OSA pathophysiology also reflected distinctive transcriptional profiling for each B-cell subpopulation. Noteworthy, identified activated networks in the two populations that were increased in OSA patients (i.e., B_Atypical and B_Memory groups, FIGS. 4G an 4F, respectively), in which expressed genes were associated with biological processes and mechanisms activated in OSA pathophysiology, such as “Oxidative Stress”, “HIF1α”, and “NF-kB signaling”. These results were in line with to those in the T-cell and myeloid subgroups, and highlight the changes in overall cellular heterogeneity induced by pediatric OSA leading to cell-specific functional pathway dysregulation in PBMCs.

### Building and Validation of a Molecular Signature for Diagnosis of Pediatric Sleep Apnea

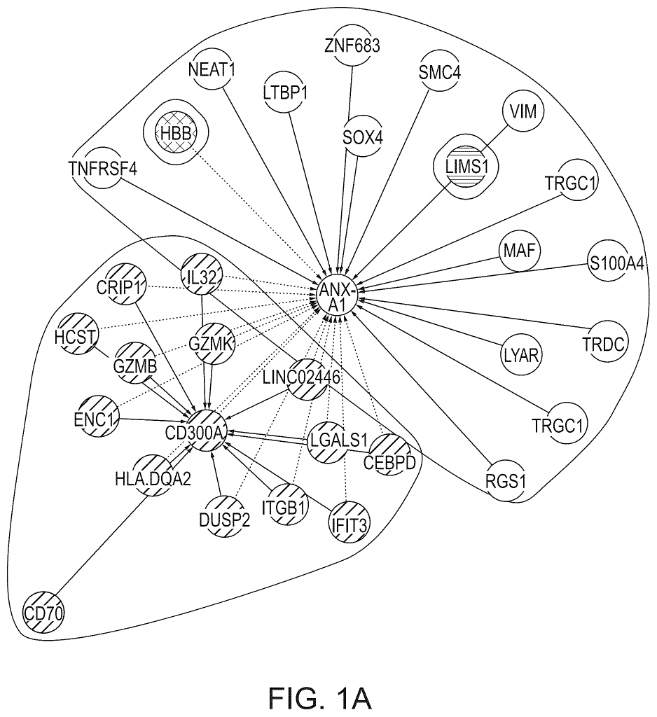

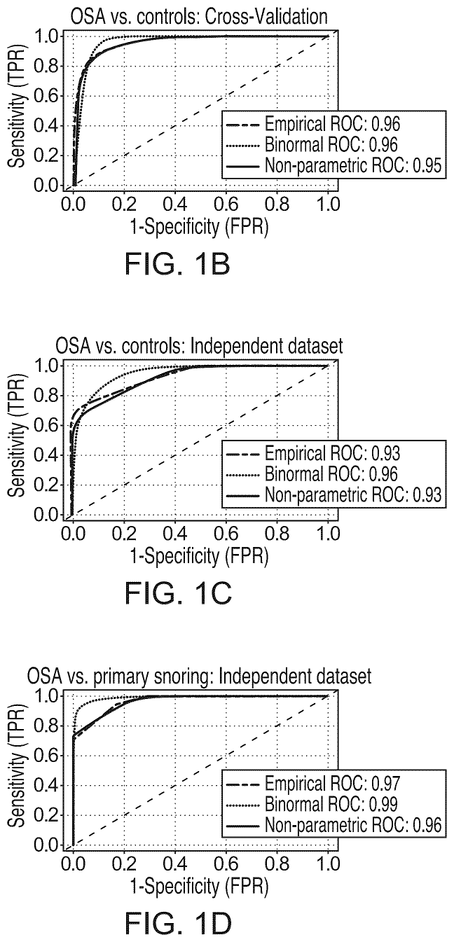

Based on the scRNAseq results, a machine learning approach was applied to build a molecular signature that discriminates between OSA patients and controls by combining markers of PBMC cellular composition with those associated with the occurrence of OSA. A molecular signature consisting of 32 genes (FIG. 1A) was extracted from each of the Seurat objects corresponding to specific cell type and clinical attributes, and combined, according to sample ID. The performance of the signature was evaluated using three statistical methods for distinguishing between OSA and Control patients in the same sample set and a cross-validation strategy. Training and testing datasets were created using a 70:30 split to generate the observed ROC curves for each statistical method (FIG. 1B). Results for the Empirical (AUC=0.96), Binormal (AUC=0.96), and Non-parametric ROC (AUC=0.95) demonstrated that the molecular signature had a very high power to discriminate between patients and controls and therefore warranted further validation studies.

Next, additional ROC-AUC analyses was performed to assess the performance of the molecular signature in a bulk RNA expression microarray-based independent PBMC sample set. The independent dataset consisted of: i) OSA patients (n=28), ii) primary snoring individuals—children who snore (e.g., have symptoms of SDB) but their sleep studies were within normal limits—(n=28), and iii) No OSA/no snoring controls (n=20). Implementation of the ROC analysis was conducted using the same computational parameters outlined for building the signature. In addition, the performance of the signature was further evaluated according to positive predictive and negative predictive values (PPV and NPV, respectively). The molecular signature showed a high performance for discriminating OSA patients from no OSA/no snoring controls, with the ROC-AUC analysis resulting in AUC of 0.93, 0.96, and 0.92 for Empirical, Binormal, and Non-parametric ROCs, respectively (FIG. 1C), with 93% and 95% PPV and NPV, respectively. Likewise, the molecular signature had a high performance to distinguish between OSA patients from primary snoring individuals (FIG. 1D) (AUCs=0.97, 0.99, and 0.96 for Empirical, Binormal, and Non-parametric ROCs, respectively; 96% PPV, and 95% NPV) and primary snoring individuals from No OSA/no snoring controls (AUC=0.95, 0.98, and 0.95 for Empirical, Binormal, and Non-parametric ROCs, respectively; 96% PPV, and 95% NPV).

Single-cell transcriptomic profiles in PBMC of OSA patients has not been studied, and these Examples illustrate the heretofore unknown OSA-induced cellular mosaicism in peripheral blood cells and their associated transcriptional landscape, providing very novel insights for understanding the impact of pediatric OSA on immune cell populations. Importantly, the study of single-cell transcriptomic profiles enabled the identification of previously undescribed PBMC cell types and the building of a molecular signature for high precision molecular diagnosis of OSA.

OSA-induced changes in cellular heterogeneity were detected in the three cell lineages in PBMCs (i.e., T-cells, Myeloid and B-cells). Besides the classical cell types in each lineage, single-cell transcriptomic profiles defined previously undescribed populations which are associated with OSA occurrence. Here two previously undescribed T-cell subpopulations (i.e., T-Cell_X1 and T-Cell_X2) were identified, whose expression profiles were mainly related to T-Cell activation, inflammation and intracellular signaling, as well as two major pathways in OSA: Hypoxia-inducible factor 1 alpha (HIF-1α) and signaling and oxidative stress. Remarkably, the percentage of cells in T-Cell_X2 significantly correlated with OSA severity, represented as increased AHI values. Specific cellular signaling pathways were activated in the T-Cell_X2 population (i.e., Phospholipase-C-, HGF-, and WNT/β-catenin signaling pathways) compared with other T-Cell populations. In addition, a monocyte subpopulation was identified whose expression profile corresponded to an IFN-primed phenotype (i.e., monocyte IFN). This is a discrete variety of classical monocytes with a stereotypic transcriptional phenotype, whose relative abundance has been linked to inflammation and promoting T cell response in tumors. Furthermore, the percentage of classical monocytes was significantly increased in OSA patients compared to controls. Although, the mechanisms for OSA-mediated activation and migration of monocytes are still undetermined, the Examples show the activation of the NF-kB signaling pathway in classical monocytes of OSA patients. NF-κB activity was increased in circulating neutrophils and monocytes in adults, which can be mitigated by therapy. Lastly, an atypical B memory cell subpopulation (i.e., B_Atypical) was detected which was significantly increased in OSA samples compared with the control group. Remarkably, comparative analysis of pathway and molecular networks revealed the activation of molecular networks associated with oxidative stress and hypoxia-inducible factor and NF-kB signaling, as observed in the T-Cell and the myeloid cell lines. Taken together these findings show the role of molecular mechanisms related to inflammation and oxidative stress, which ultimately will define unique cellular phenotypes in PBMCs as a response to OSA and its physiological components.

Polysomnography (PSG) represents the golden standard for the SDB in adults and children. PSG requires staying overnight in a laboratory, where sleep, respiratory and neurological parameters are monitored by a trained technician. PSG results are complex, and their interpretation require evaluation by a highly trained sleep physician. As such, PSG is an expensive medical procedure which represents a significant burden for the patient, insurers, and the health system at large. Therefore, there is a need for molecular diagnostic methods which can simplify OSA diagnosis, providing an accurate, quick, and affordable method.

These Examples identified a molecular signature consisting of 32 genes, which can distinguish OSA patients from controls with high precision and high positive and negative predictive value in an independent microarray-based “bulk” RNA expression dataset. Hence, this molecular signature can also be assessed in standard RNA samples, enabling the deconvolution of “bulk” RNA signal without the need for pre-analytical cell separation and making the assay amenable for application in a clinical setting. Bulk expression can be detected with single cell RNA sequencing, polymerase chain reaction (e.g., quantitative real-time PCR), targeted RNA sequencing, microarray analysis, and combinations thereof, as described herein.

The Examples further identified specific cell types in subjects having OSA. Additionally, differential gene expression for each cell type can be used to identify a subject having OSA.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

### Documents

The following documents are incorporated by reference to the extent that methods and compositions disclosed are used in practice of the present disclosure.
1. Young, T., Peppard, P. E. & Gottlieb, D. J. Epidemiology of obstructive sleep apnea: a population health perspective. Am J Respir Crit Care Med 165, 1217-1239 (2002).
2. Benjafield, A. v. et al. Estimation of the global prevalence and burden of obstructive sleep apnoea: a literature-based analysis. The Lancet Respiratory Medicine 7, 687-698 (2019).
3. DelRosso, L. M. Epidemiology and Diagnosis of Pediatric Obstructive Sleep Apnea. Current Problems in Pediatric and Adolescent Health Care 46, 2-6 (2016).
4. Dempsey, J. A., Veasey, S. C., Morgan, B. J. & O'Donnell, C. P. Pathophysiology of sleep apnea. Physiological Reviews 90, 47-112 (2010).
5. Gozal, D. Sleep, sleep disorders and inflammation in children. Sleep Medicine 10, S12-6 (2009).
6. Lumeng, J. C. & Chervin, R. D. Epidemiology of pediatric obstructive sleep apnea. Proc Am Thorac Soc 5, 242-252 (2008).
7. Vrints, H. et al. Cardiovascular mechanisms and consequences of obstructive sleep apnoea. Acta Clinica Belgica 68, 169-178 (2013).
8. Kheirandish-Gozal, L., Yoder, K., Kulkarni, R., Gozal, D. & Decety, J. Preliminary functional MRI neural correlates of executive functioning and empathy in children with obstructive sleep apnea. Sleep 37, 587-592 (2014).
9. Kheirandish, L. & Gozal, D. Neurocognitive dysfunction in children with sleep disorders. Developmental Science 9, 388-399 (2006).
10. Isaiah, A., Ernst, T., Cloak, C. C., Clark, D. B. & Chang, L. Associations between frontal lobe structure, parent-reported obstructive sleep disordered breathing and childhood behavior in the ABCD dataset. Nature Communications 12, 2205 (2021).
11. Kheirandish-Gozal, L. & Gozal, D. Obstructive sleep apnea and inflammation: Proof of concept based on two illustrative cytokines. International Journal of Molecular Sciences 20, 459 (2019).
12. Ke, D. et al. Enhanced interleukin-8 production in mononuclear cells in severe pediatric obstructive sleep apnea. Allergy, Asthma and Clinical Immunology 15, 23 (2019).
13. Kheirandish-Gozal, L. & Gozal, D. Pediatric OSA Syndrome Morbidity Biomarkers: The Hunt Is Finally On! Chest vol. 151 500-506 (Elsevier Inc, 2017).
14. Polotsky, V. Y., Bevans-Fonti, S., Grigoryev, D. N. & Punjabi, N. M. Intermittent hypoxia alters gene expression in peripheral blood mononuclear cells of healthy volunteers. PLoS ONE 10, e0144725 (2015).
15. Chen, Y. C. et al. Genome-wide gene expression array identifies novel genes related to disease severity and excessive daytime sleepiness in patients with obstructive sleep apnea. PLoS ONE 12, e0176575 (2017).
16. Lin, S. W., Tsai, C. N., Lee, Y. S., Chu, S. F. & Chen, N. H. Gene expression profiles in peripheral blood mononuclear cells of Asian obstructive sleep apnea patients. Biomed J 37, 60-70 (2014).
17. Khalyfa, A. et al. Genome-wide gene expression profiling in children with non-obese obstructive sleep apnea. Sleep Med 10, 75-86 (2009).
18. Kheirandish-Gozal, L. et al. Nitric oxide production by monocytes in children with OSA and endothelial dysfunction. Clin Sci (Lond) 127, 323-330 (2014).
19. Tan, H.-L. et al. Alterations in Circulating T-Cell Lymphocyte Populations in Children with Obstructive Sleep Apnea. Sleep 36, 913-922 (2013).
20. de la Pena, M. et al. Endothelial Function and Circulating Endothelial Progenitor Cells in Patients with Sleep Apnea Syndrome. Respiration 76, 28-32 (2008).
21. Kheirandish-Gozal, L., Bhattacharjee, R., Kim, J., Clair, H. B. & Gozal, D. Endothelial progenitor cells and vascular dysfunction in children with obstructive sleep apnea. American Journal of Respiratory and Critical Care Medicine 182, 92-97 (2010).
22. Stavnezer, J., Guikema, J. E. J. & Schrader, C. E. Mechanism and regulation of class switch recombination. Annu Rev Immunol 26, 261-292 (2008).
23. Sutton, H. J. et al. Atypical B cells are part of an alternative lineage of B cells that participates in responses to vaccination and infection in humans. Cell Rep 34, (2021).
24. Holla, P. et al. Shared transcriptional profiles of atypical B cells suggest common drivers of expansion and function in malaria, HIV, and autoimmunity. Sci Adv 7, (2021).
25. Sullivan, R. T. et al. FCRL5 Delineates Functionally Impaired Memory B Cells Associated with Plasmodium falciparum Exposure. PLoS Pathog 11, (2015).
26. Aldridge, S. & Teichmann, S. A. Single cell transcriptomics comes of age. Nat Commun 11, (2020).
27. Heath, J. R., Ribas, A. & Mischel, P. S. Single-cell analysis tools for drug discovery and development. Nature Reviews Drug Discovery vol. 15 204-216 (2016).
28. Mulder, K. et al. Cross-tissue single-cell landscape of human monocytes and macrophages in health and disease. Immunity 54, 1883-1900.e5 (2021).
29. Fan, C., Huang, S., Xiang, C., An, T. & Song, Y. Identification of key genes and immune infiltration modulated by CPAP in obstructive sleep apnea by integrated bioinformatics analysis. PLoS One 16, (2021).
30. Chen, Y. C. et al. Genome-wide gene expression array identifies novel genes related to disease severity and excessive daytime sleepiness in patients with obstructive sleep apnea. PLoS One 12, (2017).
31. Turnbull, C. D. et al. Transcriptomics Identify a Unique Intermittent Hypoxia-mediated Profile in Obstructive Sleep Apnea. Am J Respir Crit Care Med 201, 247-250 (2020).
32. Cofta, S. et al. Oxidative Stress Markers and Severity of Obstructive Sleep Apnea. Adv Exp Med Biol 1222, 27-35 (2019).
33. Gabryelska, A., Karuga, F. F., Szmyd, B. & Bialasiewicz, P. HIF-1α as a Mediator of Insulin Resistance, T2DM, and Its Complications: Potential Links With Obstructive Sleep Apnea. Front Physiol 11, (2020).
34. Yuan, G., Nanduri, J., Khan, S., Semenza, G. L. & Prabhakar, N. R. Induction of HIF-1alpha expression by intermittent hypoxia: involvement of NADPH oxidase, Ca2+ signaling, prolyl hydroxylases, and mTOR. J Cell Physiol 217, 674-685 (2008).
35. Polak, J. et al. Intermittent hypoxia impairs glucose homeostasis in C57BL6/J mice: Partial improvement with Cessation of the exposure. Sleep 36, 1483-1490 (2013).
36. He, Q. et al. Effects of varying degrees of intermittent hypoxia on proinflammatory cytokines and adipokines in rats and 3T3-L1 adipocytes. PLoS ONE 9, e86326 (2014).
37. Olea, E. et al. Intermittent hypoxia and diet-induced obesity: Effects on oxidative status, sympathetic tone, plasma glucose and insulin levels, and arterial pressure. Journal of Applied Physiology 117, 706-719 (2014).
38. Olefsky, J. M. & Glass, C. K. Macrophages, inflammation, and insulin resistance. Annual Review of Physiology 72, 219-246 (2009).
39. Gileles-Hillel, A. et al. Early intermittent hypoxia induces proatherogenic changes in aortic wall macrophages in a murine model of obstructive sleep apnea. American Journal of Respiratory and Critical Care Medicine 190, 958-961 (2014).
40. Bouhlel, M. A. et al. PPARy Activation Primes Human Monocytes into Alternative M2 Macrophages with Anti-inflammatory Properties. Cell Metabolism 6, 137-143 (2007).
41. Nair, D., Dayyat, E. A., Zhang, S. X., Wang, Y. & Gozal, D. Intermittent hypoxia-induced cognitive deficits are mediated by NADPH oxidase activity in a murine model of Sleep Apnea. PLoS ONE 6, e19847 (2011).
42. Row, B. W., Liu, R., Xu, W., Kheirandish, L. & Gozal, D. Intermittent Hypoxia Is Associated with Oxidative Stress and Spatial Learning Deficits in the Rat. American Journal of Respiratory and Critical Care Medicine 167, 1548-1553 (2003).
43. Row, B. W. et al. Platelet-activating factor receptor-deficient mice are protected from experimental sleep apnea-induced learning deficits. J Neurochem 89, 189-96 (2004).
44. Shan, X. et al. Manganese superoxide dismutase protects mouse cortical neurons from chronic intermittent hypoxia-mediated oxidative damage. Neurobiol Dis 28, 206-15 (2007).
45. Xu, W. et al. Increased oxidative stress is associated with chronic intermittent hypoxia-mediated brain cortical neuronal cell apoptosis in a mouse model of sleep apnea. Neuroscience 126, 313-323 (2004).
46. Jun, J. et al. Intermittent hypoxia has organ-specific effects on oxidative stress. American Journal of Physiology-Regulatory Integrative and Comparative Physiology 295, R1274-81 (2008).
47. Li, R. C. et al. Exogenous growth hormone attenuates cognitive deficits induced by intermittent hypoxia in rats. Neuroscience 196, 237-250 (2011).
48. Liu, F., Liu, T. W. & Kang, J. The role of NF-κB-mediated JNK pathway in cognitive impairment in a rat model of sleep apnea. Journal of Thoracic Disease 10, 6921-6931 (2018).
49. Gozal, D. & Kheirandish-Gozal, L. Cardiovascular morbidity in obstructive sleep apnea: oxidative stress, inflammation, and much more. Am J Respir Crit Care Med 177, 369-375 (2008).
50. McNicholas, W. T. Obstructive Sleep Apnea and Inflammation. Progress in Cardiovascular Diseases 51, 392-399 (2009).
51. de Lima, F. F. F., Mazzotti, D. R., Tufik, S. & Bittencourt, L. The role inflammatory response genes in obstructive sleep apnea syndrome: a review. Sleep and Breathing 20, 331-338 (2016).
52. Deng, Y. et al. P2X7 Receptor Antagonism Attenuates the Intermittent Hypoxia-induced Spatial Deficits in a Murine Model of Sleep Apnea Via Inhibiting Neuroinflammation and Oxidative Stress. Chinese Medical Journal 128, 2168-2175 (2015).
53. Pan, Y. Y. et al. Altered Wnt Signaling Pathway in Cognitive Impairment Caused by Chronic Intermittent Hypoxia: Focus on Glycogen Synthase Kinase-3β and β-catenin. Chin Med J (Engl) 129, 838-845 (2016).
54. Mulder, K. et al. Cross-tissue single-cell landscape of human monocytes and macrophages in health and disease. Immunity 54, 1883-1900.e5 (2021).
55. Zilionis, R. et al. Single-Cell Transcriptomics of Human and Mouse Lung Cancers Reveals Conserved Myeloid Populations across Individuals and Species. Immunity 50, 1317-1334.e10 (2019).
56. Greenberg, H. et al. Chronic intermittent hypoxia activates nuclear factor-kappaB in cardiovascular tissues in vivo. Biochem Biophys Res Commun 343, 591-596 (2006).
57. Htoo, A. K. et al. Activation of nuclear factor kappaB in obstructive sleep apnea: a pathway leading to systemic inflammation. Sleep Breath 10, 43-50 (2006).
58. Khurana, S., Soda, N., Shiddiky, M. J. A., Nayak, R. & Bose, S. Current and future strategies for diagnostic and management of obstructive sleep apnea. Expert Rev Mol Diagn 21, 1287-1301 (2021).
59. Byrnes, S. A. & Weigl, B. H. Selecting analytical biomarkers for diagnostic applications: a first principles approach. Expert Rev Mol Diagn 18, 19-26 (2018).
60. de Luca Canto, G. et al. Biomarkers associated with obstructive sleep apnea: A scoping review. Sleep Med Rev 23, 28-45 (2015).
61. Bencharit, S., Redenz, R. G., Brody, E. R. & Chiang, H. Salivary biomarkers associated with obstructive sleep apnea: a systematic review. Expert Rev Mol Diagn 21, 223-233 (2021).
62. Kang, K.-T., Lee, P.-L., Weng, W.-C. & Hsu, W.-C. Body weight status and obstructive sleep apnea in children. Int J Obes (Lond) 36, 920-924 (2012).
63. Ward, C. M., To, T. H. & Pederson, S. M. ngsReports: a Bioconductor package for managing FastQC reports and other NGS related log files. Bioinformatics 36, 2587-2588 (2020).

1. A method of diagnosing obstructive sleep apnea in a subject having or suspected of having obstructive sleep apnea, the method comprising:
providing a biological sample from the subject;
determining a gene expression level of CD70, HCST, CRIP1, IL 32, ENC1, GZMB, GZMK, HLA.DQA2, CD300A, LINC02446, DUSP2, ITGB1, LGALS1, IFIT3, CEBPD, TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof, and
diagnosing obstructive sleep apnea in the subject if there is a measurable change in the expression level of the CD70, the HCST, the CRIP1, the IL 32, the ENC1, the GZMB, the GZMK, the HLA.DQA2, the CD300A, the LINC02446, the DUSP2, the ITGB1, the LGALS1, the IFIT3, the CEBPD, the TNFRSF4, the HBB, the NEAT1, the LTBPL the ZNF683, the SOX4, the SMC4, the LMS1, the VIM. the TRGCL, the MAF, the S100A4, the LYAR, the TRDC, the TRGC2, the RGS1, the ANXA1, and combinations thereof, as compared to a reference expression of CD70, HCST, CRIP1, IL 32, ENC1, GZMB, GZMK, HLA.DQA2, CD300A, LINC02446, DUSP2, ITGB1, LGALS1, IFIT3, CEBPD, TNFRSF4, HBB, NEAT1, LTBPL ZNF683, SOX4, SMC4, LMS1, VIM. TRGCL, MAF, S100A4, LYAR, TRDC, TRGC2, RGS1, ANXA1, and combinations thereof, obtained from a subject who does not have obstructive sleep apnea.

2. The method of claim 1, wherein the subject having or suspected of having obstructive sleep apnea is a human subject.

3. The method of claim 1, wherein the human subject is selected from a pediatric human subject, an adolescent human subject, and an adult human subject.

4. The method of claim 1, wherein the biological sample is a peripheral blood mononuclear cell sample.

5. The method of claim 1, wherein the biological sample is circulating RNA.

6. The method of claim 1, further comprising performing Polysomnography (PSG) on the subject having or suspected of having obstructive sleep apnea.

7. The method of claim 1, further comprising determining Apnea Hypopnea Index for the subject having or suspected of having obstructive sleep apnea.

8. The method of claim 1, further comprising administering a treatment for obstructive sleep apnea to the subject diagnosed as having obstructive sleep apnea.

9. The method of claim 8. wherein the treatment is selected from a lifestyle change, positive airway pressure, an oral appliance, surgery, and combinations thereof.

10. A method of treating a subject having or suspected of having obstructive sleep apnea, the method comprising:
providing a biological sample from the subject;
determining a gene expression level of CD70, HCST, CRIP1, IL 32, ENC1, GZMB, GZMK, HLA.DQA2. CD300A, LINC02446, DUSP2, ITGB1, LGALS1, IFIT3, CEBPD, TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof,
diagnosing obstructive sleep apnea in the subject if there is a measurable change in the expression level of the CD70, the HCST, the CRIP1, the IL 32, the ENC1, the GZMB, the GZMK, the HLA.DQA2, the CD300A, the LINC02446, the DUSP2, the ITGB1, the LGALS1, the IFIT3, the CEBPD, the TNFRSF4, the HBB, the NEAT1, the LTBP1, the ZNF683, the SOX4, the SMC4, the LMS1, the VIM, the TRGC1, the MAF, the S100A4, the LYAR, the TRDC, the TRGC2, the RGS1, the ANXA1, and combinations thereof, as compared to a reference expression of CD70, HCST, CRIP1, IL 32, ENC1, GZMB, GZMK, HLA.DQA2, CD300A, LINC02446, DUSP2, ITGBL, LGALS1, IFIT3, CEBPD, TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof, obtained from a subject who does not have obstructive sleep apnea; and
treating the subject diagnosed as having obstructive sleep apnea by administering a treatment for obstructive sleep apnea to the subject diagnosed as having obstructive sleep apnea.

11. The method of claim 10, wherein the treatment is selected from a lifestyle change, positive airway pressure, an oral appliance, and surgery.

12. A biomarker panel for diagnosing obstructive sleep apnea in a subject in need thereof comprising: CD70, HCST, CRIP1, IL 32, ENC1, GZMB, GZMK, HLA.DQA2, CD300A, LINC02446, DUSP2, ITGB1, LGALS1, IFIT3, CEBPD, TNFRSF4, HBB, NEAT1, LTBP1, ZNF683, SOX4, SMC4, LMS1, VIM, TRGC1, MAF, S100A4, LYAR, TRDC, TRGC2, RGS1, ANXA1, and combinations thereof.

13. (canceled)
